(19) United States
(12) Patent Application Publication
(10) Pub. No.: US 2019/0167936 A1
(43) Pub. Date: Jun. 6, 2019

(54) Nasal Cannula Clamping and Patient Monitoring System

(71) Applicant: Roger Cameron Love, Show Low, AZ (US)

(72) Inventor: Roger Cameron Love, Show Low, AZ (US)

(21) Appl. No.: 16/184,901

(22) Filed: Nov. 8, 2018

Related U.S. Application Data
(63) Continuation-in-part of application No. 15/830,515, filed on Dec. 4, 2017, now abandoned.

(51) Int. Cl. A61M 16/06; A61M 16/08; A61M 16/00

(52) U.S. Cl. CPC A61M 16/0666; A61M 16/0875; A61M 2025/024; A61M 16/0051; A61M 16/024; A61M 16/0833

(57) ABSTRACT

A nasal cannula clamping system which holds a nasal cannula in position on a patient. The nasal cannula clamping system includes a nasal cannula clamp assembly having a clamp body and a clamp block grasping nasal cannula tubes in two non-round parallel passages/grooves which securely clamps the two tubes under the chin while not compromising the distribution of medication prescribed by the attending physician. The nasal cannula tubes are sandwiched between the clamp body and clamp block which also contains the non-round parallel passages/grooves.

### CROSS-REFERENCE TO RELATED APPLICATIONS

The present application is a Continuation In Part of U.S. Patent Application Ser. No. 15/830,515 filed Dec. 4, 2017, which application is incorporated in its entirety herein by reference.

### BACKGROUND OF THE INVENTION

The present invention relates to a nasal cannula and in particular to a parabolic clamping grooves system and monitor for use with the nasal cannula.

Nasal cannulas are connected to a patient to provide a flow of gases into the patient's nose. The source of the treatment and/or medication to the patient through the nasal cannula are prescribed along with the flow rate of gas such as oxygen, room air, and any gas mixture, etc. in Liters Per Minute (LPM) by an attending physician and connected to the nasal cannula input line to the patient and attended to by a physician, nursing staff (if hospitalized), patient/family (if at home), etc.

Known cannula clamps grasp round tubes between circular halves. Because the tubes and the clams both have round cross-sections, the clamps must have a smaller inside diameter than the tubing outside diameter to grasping the tubing. As a result, the clamps must at least partially collapse the tubing to prevent the tubing from slipping inside the clamp, and thereby restrict the flow of gases to the patient.

### BRIEF SUMMARY OF THE INVENTION

The present invention addresses the above and other needs by providing a nasal cannula clamping system which holds a nasal cannula in position on a patient. The nasal cannula clamping system includes a nasal cannula clamp assembly having a clamp body and a clamp block grasping nasal cannula tubes in two non-round parallel passages/grooves which securely clamps the two tubes under the chin while not compromising the distribution of medication prescribed by the attending physician. The nasal cannula tubes are sandwiched between the clamp body and clamp block which also contains the non-round parallel passages/grooves.

In accordance with one aspect of the invention, there is provided a nasal cannula clamping system providing enhanced flow to a patient. In many cases, a nasal cannula is worn while sleeping. If the nasal cannula moves sufficiently during sleep, the patient is awakened, losing important sleep time, and if the flow is interrupted, the patient may experience health risks. Also, the patient can be monitored in regard to certain vital health aspects without disturbing or wakening said patient via Wi-Fi wireless connection. The nasal cannula clamping system is also completely self contained without the need for peripheral equipment either attached to the individual or stand alone such as bench top analytical equipment for analyzing breathing habits, O2 to blood ratio, heart rate to name a few and is all handled by the self contained unit via Wi-Fi.

In accordance with still another aspect of the invention, there is provided a nasal cannula clamping system including a lock screw tightening a clamp block against a clamp body to hold nasal cannula tubes in place under the chin. An optional independent flow sensor can be placed in between the nasal cannula connection and the input line from a gas source. The flow sensor is only to detect the flow of whatever gas is passing through the source tube and serves no other function. If the flow sensor unit is attached and a flow is not detected, an alarm is provided. The alarm may be visual using the LED, an audible alarm, a vibration alarm, or a wireless signal which may be employed to notify the attending individual of an issue with flow. The flow sensor unit is not required for full functionality of the main body of the system and as an independent unit which simply unplugs from the flow line.

In accordance with yet another aspect of the invention, there is provided a nasal cannula clamp assembly including a circuit board, batteries, an activator switch, and an indicator. The circuitry processes data from the independent flow sensor (if installed) to monitor breathing and provides an alarm when a flow is not detected the system goes into standby mode until the flow issue is resolved. The alarm may be visual using the LED, audible, a vibration, or a wireless signal. The wireless signal may be transmit via Wi-Fi or other wireless protocols, and may be in the form of a text message.

In accordance with another aspect of the invention, there is provided a nasal cannula clamp assembly having clamps having a non-round interior cross-section. The interior cross-section may be parabolic, elliptical, oval, or any non-round shape. The clamps distort tubing in the clamps to grasp the tubing, and do not substantially collapse the tubing, thus not compromising the distribution of medication prescribed by the attending physician to the patient.

### BRIEF DESCRIPTION OF THE SEVERAL VIEWS OF THE DRAWING

The above and other aspects, features and advantages of the present invention will be more apparent from the following more particular description thereof, presented in conjunction with the following drawings wherein:

FIG. 1 shows a patient wearing a nasal cannula system according to the present invention.

FIG. 1A Shows the standard off shelf Nasal Cannula with the nasal cannula system and independent flow sensor according to the present invention.

FIG. 2A shows a front view of the nasal cannula clamping system according to the present invention.

FIG. 2B shows a rear view of the nasal cannula clamping system according to the present invention.

FIG. 2C shows a side view of the nasal cannula clamping system according to the present invention.

FIG. 2D shows a top view of the nasal cannula clamping system according to the present invention.

FIG. 3A is a front and left side view of the clamping system according to the present invention.

FIG. 3B is a rear and left side view of the nasal cannula clamping system according to the present invention.

FIG. 4 is an exploded view of the nasal cannula clamping system according to the present invention.

FIG. 4A shows non-round passages according to the present invention.

FIG. 5A is a front and right side perspective view of the nasal cannula clamp assembly according to the present invention.

FIG. 5B is a rear and right side perspective view of the nasal cannula clamp assembly according to the present invention.

FIG. 5C is a bottom, front, and right side perspective view of the nasal cannula clamp assembly according to the present invention.

FIG. 5D is a bottom and right side perspective view of the nasal cannula clamp assembly according to the present invention.

FIG. 6A is a front view of an independent flow sensor assembly of the nasal cannula clamping system according to the present invention.

FIG. 6B is a rear and right side view of the independent flow sensor assembly of the nasal cannula clamping system according to the present invention.

FIG. 6C is an exploded view of the independent flow sensor assembly of the nasal cannula clamping system according to the present invention.

FIG. 7 is a front view of the Nasal cannula clamping system according to the present invention with a biometric ear lobe clip sensor and independent flow sensor attached.

FIG. 8A shows the nasal cannula clamping system including a flow sensor according to the present invention.

FIG. 8B shows the nasal cannula clamping system without the flow sensor according to the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

Where the terms “about” or “generally” are associated with an element of the invention, it is intended to describe a feature's appearance to the human eye or human perception, and not a precise measurement.

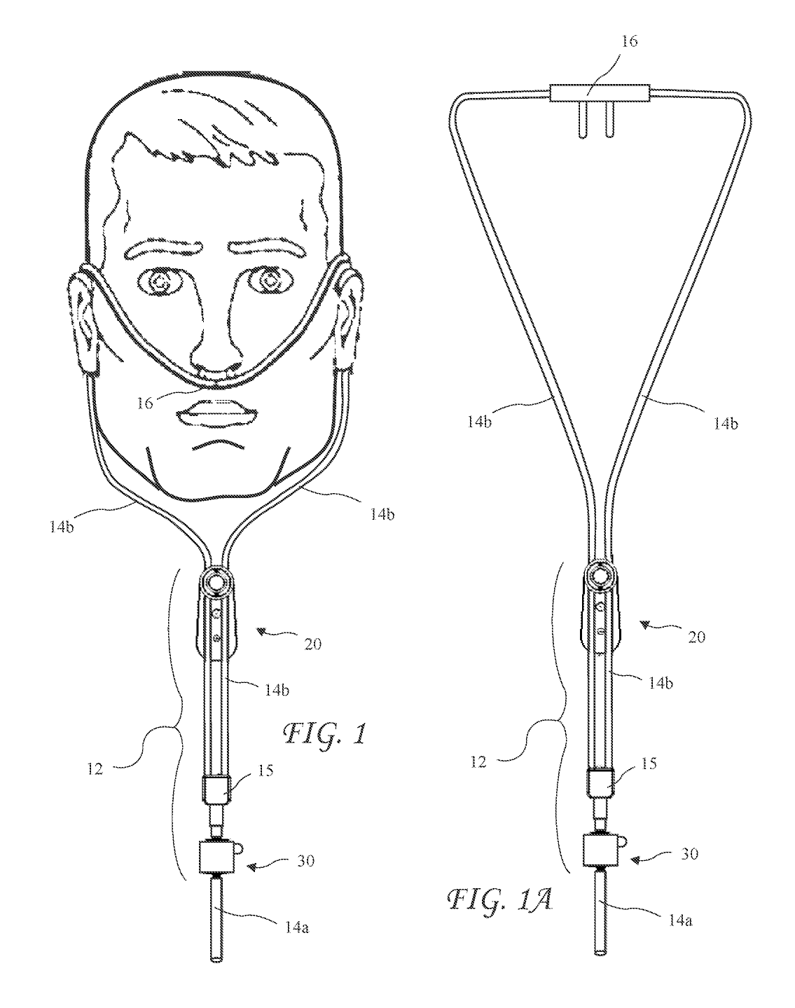

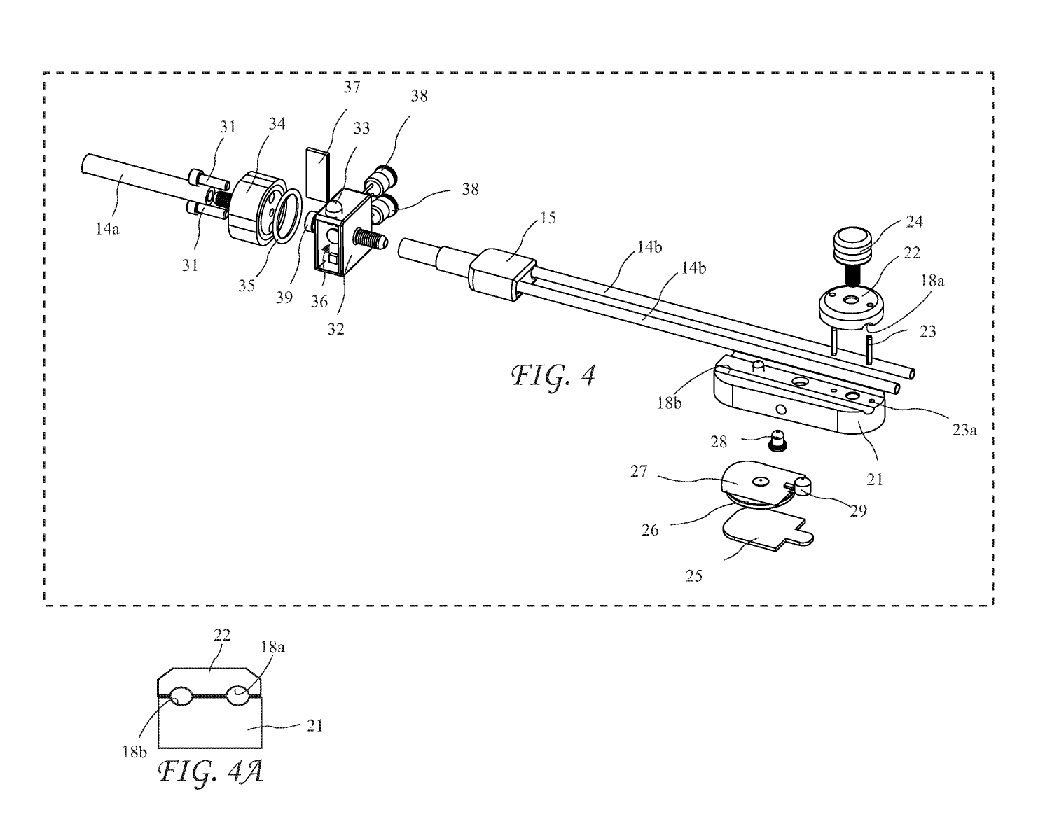

A patient 10 wearing a nasal cannular 14 (see FIG. 1A) secured by a nasal cannular clamping system 12 according to the present invention is shown in FIG. 1. The nasal cannular clamping system 12 is attached via non-round parallel passages/grooves 18a and 18b (see FIG. 4A) over second cannula tubes 14b under a patient chin, holding a nose piece 16 and over the ear tubes 14b of the cannula 14 in place. The cannula clamping system 12 includes the cannula clamp assembly 20, the standard off the shelf cannula 14, and a stand alone flow sensor unit 30 confirming flow to the cannula from a main source is shown in FIG. 1A.

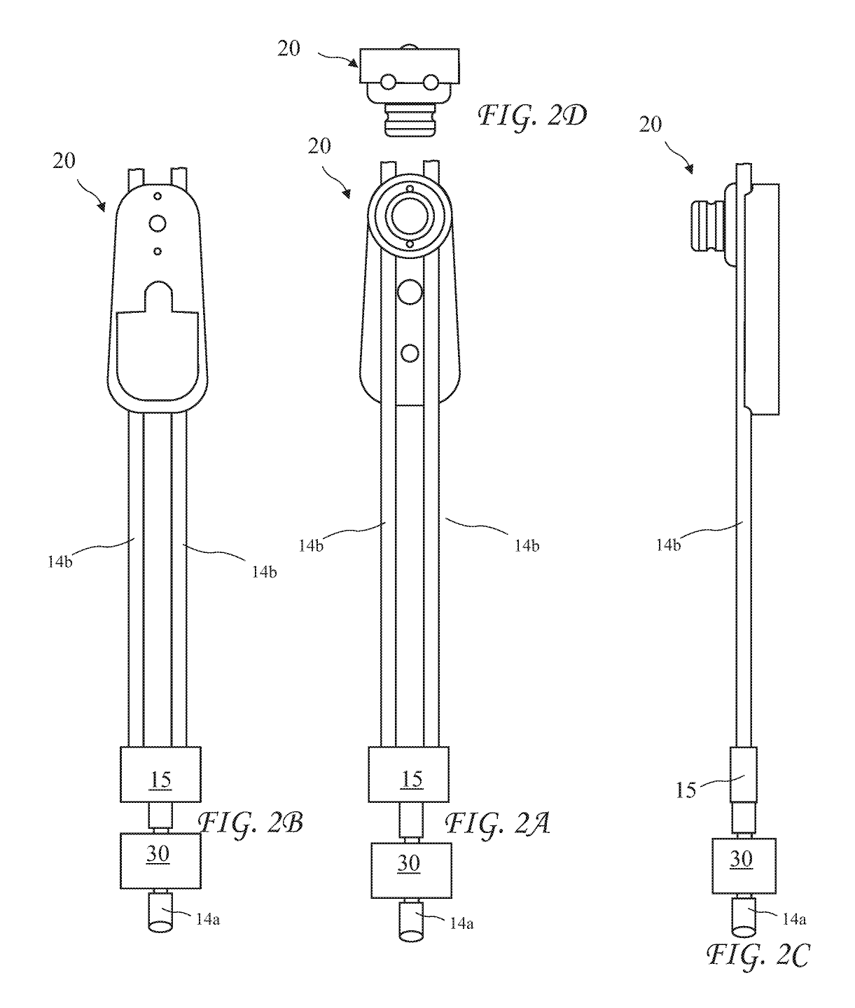

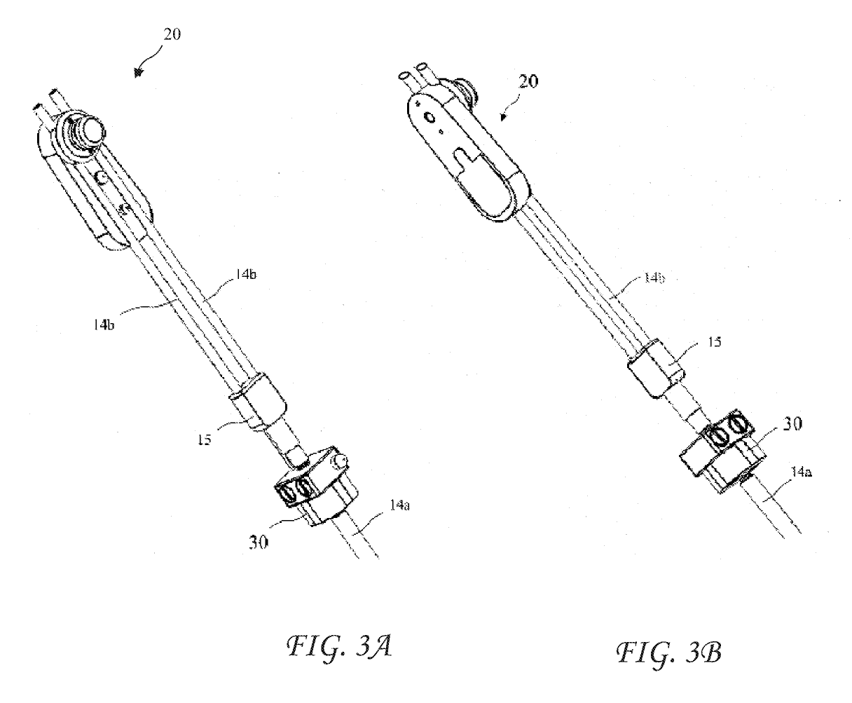

A front view of the Cannula clamping system 12 is shown in FIG. 2A, a rear view of the Cannula clamping system 12 is shown in FIG. 2B, a side view of the Cannula clamping system 12 is shown in FIG. 2C, and a top view of the Cannula clamping system 12 is shown in FIG. 2A. A front and left side view of the Cannula clamping system 12 is shown in FIG. 3A and a rear and left side view of the Cannula clamping system 12 is shown in FIG. 3B.

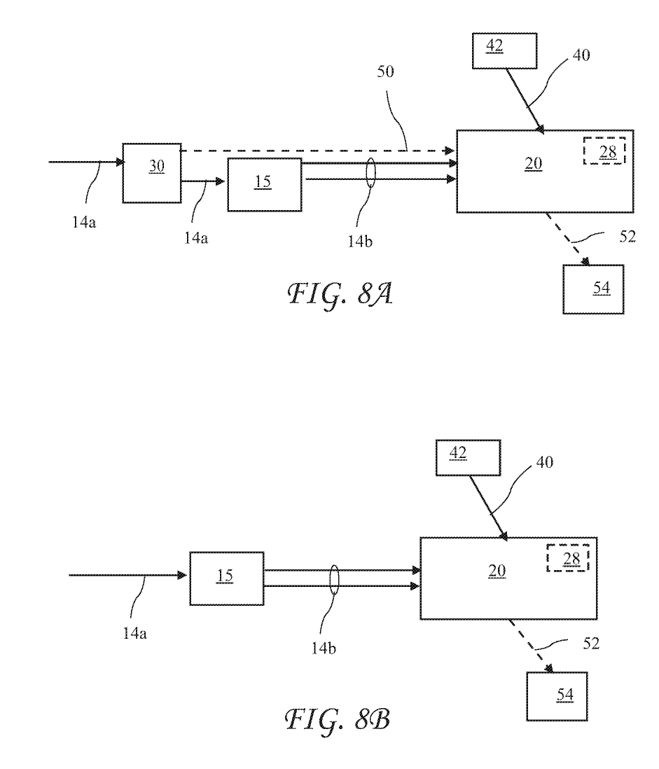

An exploded view of the nasal cannula clamping system 12 is shown in FIG. 4. The Cannula clamp assembly 20 includes a clamp body 21 and a clamp block 22 for grasping the Cannula tubes 14b. A fastener, for example a lock screw) 24 passes through the block 22 and threads into the clamp body 21 to draw the clamp block 22, guided by pins 23 in pin holes 23a, against the clamp body 21, to grasp the Cannula tubes 14b between tube passages 18a and 18b. A circuit board 27 and battery 26 reside in the clamp body 21 covered by a cover 25. An indicator 29 is controlled by the circuit board 27 and a switch 28 controls the circuit board 27. The indicator may be an optical indicator (e.g., and LED), an audio indicator, or a vibrating indicator, or a wireless signal 52 sent to device 54 (see FIGS. 8A, 8B). The wireless signal 52 may be transmit via WiFi or other wireless protocols, and may be in the form of a text message.

A cross-sectional view of the clamp block 22 against the clamp body 21 is shown in FIG. 4A. The clamp block 22 includes non-round passages 18a and the clamp body 21 includes non-round passages 18b. The non-round passages 18a and 18b preferably and a width slightly greater than the diameter of the tubes 14a and 14b, a depth less than the radius of the tubes 14a and 14b. For example passages 18a and 18b for a standard adult cannula are preferably 0.129+0.001 or −0.002 inches wide and 0.059+0.0020 or −0.0010 inches deep. Passages 18a and 18b for a standard child cannula are preferably 0.088+0.0005 or −0.0010 inches wide and 0.040+0.0005 or −0.0010 inches deep. For other cannula tubes, the passages 18a and 18b are preferably between 0.005 and 0.002 inches wider than the tube diameter and between 0.0015 and 0.0045 shallower than the radius of the tube. The passages 18a and 18b are preferably smooth and more preferably are parabolic, elliptical, or oval shaped.

The optional flow sensor assembly 30 includes a sensor body 32, a sensor cover 34 held against the sensor body 32 by screws 31, and an O-Ring 35 between the sensor cover 34 and sensor body 32. A flow sensor 39 resides in the sensor body 32 and a sensor circuit 36 and LED 33 receive power from batteries 38 internally wired to the circuit board 36 on the flow sensor unit to process signals from the flow sensor 39 and is covered by a circuit cover 37.

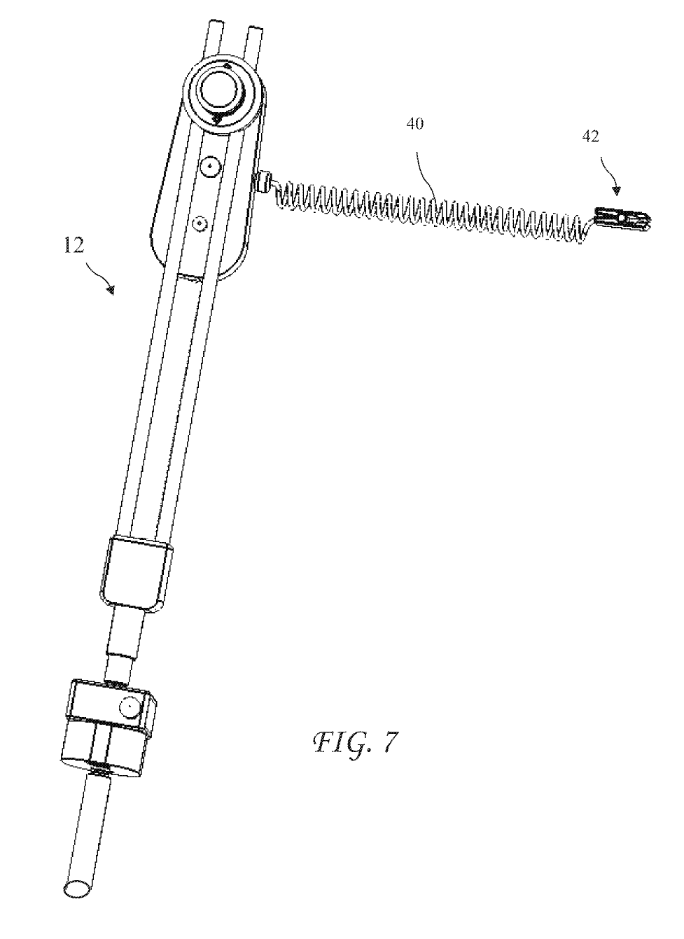

The circuit board 27 processes data from the biometric sensors 42 (see FIG. 7) to provide patient monitoring data and alerts. The patient monitoring data may be carried by led 40 or transmitted via WiFi or other wireless protocols directly to the WiFi or wireless network. The alerts may also be provided by the indicator multi color LED 29.

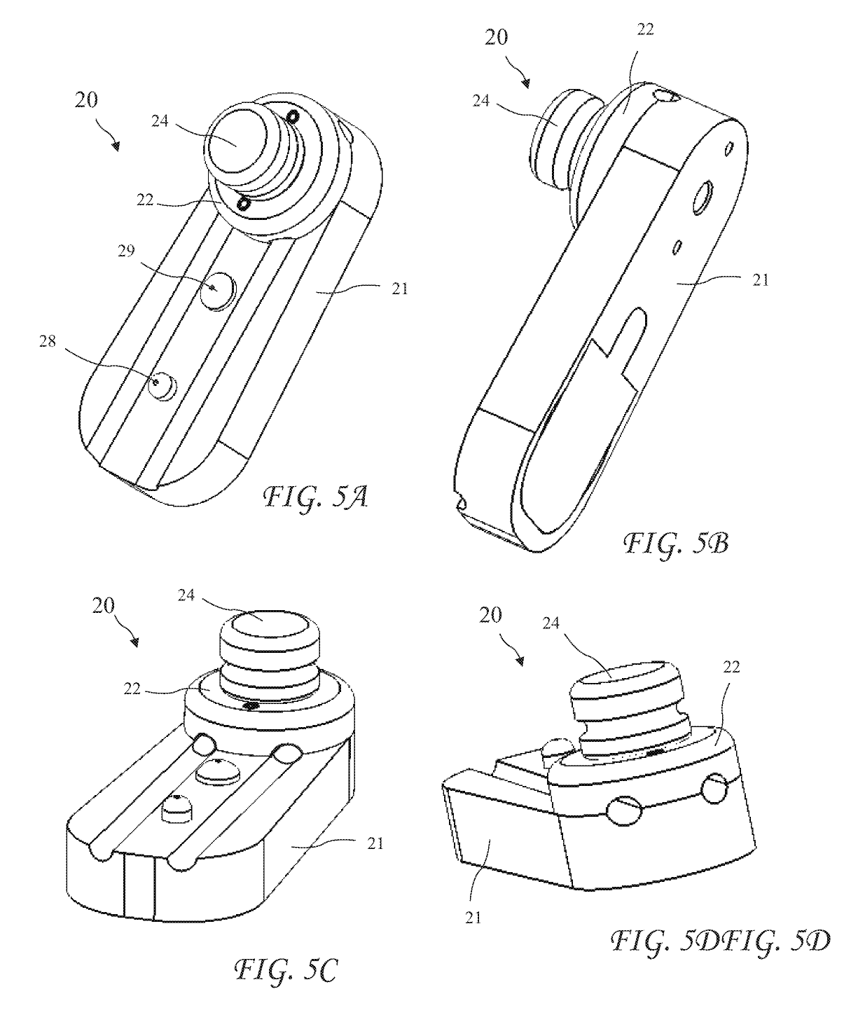

A front and right side perspective view of the cannula clamp assembly 20 is shown in FIG. 5A, a rear and right side perspective view of the cannula clamp assembly 20 is shown in FIG. 5B, a bottom, front, and right side perspective view of the cannula clamp assembly 20 is shown in FIG. 5C, and a bottom and right side perspective view of the cannula clamp assembly 20 is shown in FIG. 5D.

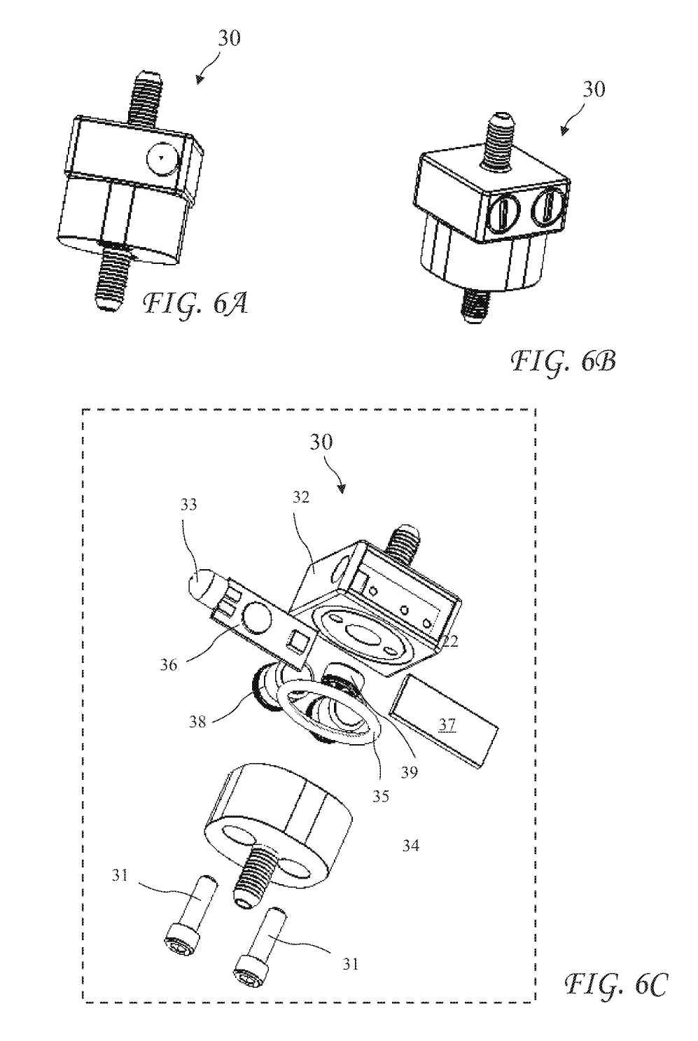

A front view of a stand alone flow sensor assembly 30 is shown in FIG. 6A, a rear and right side view of the flow sensor assembly 30 is shown in FIG. 6B, and an exploded view of the flow sensor assembly 30 is shown in FIG. 6C.

A front view of the nasal cannular clamping system 12 with a biometric sensor 42 attached by a lead 40 is shown in FIG. 7. The biometric sensor 42 may be a heart rate Beats Per Minute (BPM) sensor, a percentage of oxygen in blood sensor, or other biometric sensor. The biometric sensor 42 may be, for example, an ear clip sensor, but may be any other type of biometric sensor. Measurements from the biometric sensor 42 are provided to the circuit board 27 and processed to provide patient monitoring and alerts, via WiFi or other wireless protocols directly to the WiFi or wireless network 54 (see FIGS. 8A and 8B).

FIG. 8A shows the nasal cannula clamping system including the flow sensor assembly 30. Wireless sensor signals 50 are transmitted from the sensor circuit 36 in the flow sensor assembly 30 to the circuit board 27 in the cannula clamp assembly 20 validating that the flow is present in the tubing 14a. The signals are processed by the circuit board 27 and a wireless signal 52 is provided to monitoring 54 by a smart phone, a tablet, a personal computer, or specialized monitoring station. If the flow sensor assembly 30 is present and no flow is detected by the flow sensor assembly 30, the signal 50 informs the circuit board 27 that no flow is present, and the wireless signal 52 is provided to monitoring 54 and the system goes to standby until the problem is corrected. If flow is detected, the system assumes a normal state of operation.

FIG. 8B shows the nasal cannula clamping system without the flow sensor assembly 30. When the flow sensor assembly 30 is not present, no signal 50 is sent and nothing occurs.

While the invention herein disclosed has been described by means of specific embodiments and applications thereof, numerous modifications and variations could be made thereto by those skilled in the art without departing from the scope of the invention set forth in the claims.

1. A nasal Cannula clamping system, comprising:
a cannula clamp assembly including:
a clamp body having two non-round cannula tubes passages extending the length of the clamp body;
a clamp block attached to the clamp body and residing over a portion of both cannula tubing recesses; and
a clamp block fastener drawing the clamp block towards the clamp body;
an independent flow sensor detecting a presence of a flow in cannula tubing;
a circuit board to process data from the independent flow sensor;
batteries electrically connected to the circuit carrying a power signal to the circuit board;
a switch controlling the circuit board; and
an indicator providing an indication of flow presence detected by the independent flow sensor, in the cannula tubing.

2. The nasal cannula clamping system of claim 1, wherein the cannula tubing recesses are generally parallel.

3. The nasal cannula clamping system of claim 1, wherein clamp block fastener is a threaded fastener.

4. The nasal cannula clamping system of claim 1, wherein the cannula tubing passages have a parabolic cross-section.

5. The nasal cannula clamping system of claim 1, wherein the cannula tubing passages have an elliptical cross-section.

6. The nasal cannula clamping system of claim 1, wherein the cannula tubing passages have an oval cross-section.

7. The nasal cannula clamping system of claim 1, wherein the cannula tubing passages have a width slightly greater than a diameter of the cannula tubing, and a depth less than a radius of the cannula tubing.

8. The nasal cannula clamping system of claim 7, wherein the cannula tubing passages are preferably between 0.005 and 0.002 inches wider than the cannula tubing diameter and between 0.0015 and 0.0045 shallower than the radius of the cannula tubing.

9. The nasal cannula clamping system of claim 7, wherein the cannula tubing passages are 0.129+0.001 or −0.002 inches wide and 0.059+0.0020 or −0.0010 inches deep

10. The nasal cannula clamping system of claim 1, wherein the independent flow sensor resides in a sensor assembly spaced apart from the cannula clamp assembly.

11. The nasal cannula clamping system of claim 10, further including a standard cannula Y connector between the independent flow sensor assembly and the clamp body, wherein:
the independent flow senor assembly receives a first flow from a main source; and
the standard cannula Y connector separates the first flow in the first cannula tube into two parallel second flows in two second cannula tubes held between the clamp body and the clamp block.

12. The nasal cannula clamping system of claim 10, wherein the circuit board, the batteries, the switch, and the indicator are attached to the cannula clamp assembly.

13. The nasal cannula clamping system of claim 1, wherein the indicator is a light, a vibrator, an audio device, or a wireless signal.

14. The nasal cannula clamping system of claim 13, wherein the wireless signal is transmitted via WiFi or wireless protocol.

15. The nasal cannula clamping system of claim 13, wherein the indicator is a light.

16. The nasal cannula clamping system of claim 1, wherein the color of the clamp body provides patient information to medical staff.

17. A method for retaining a nasal cannula, the method comprising:
positioning two second cannula tubes of the nasal cannula on a patient, the two second cannula tubes connected to a standard cannula Y ;
connecting a first cannula tube between a main source and the standard cannula Y;
resting two portions of the second cannula tubes in first non-round cannula tubing recesses of a clamp body of a nasal cannula clamping system;
positioning a clamp block having two second non-round cannula tubing recesses over the two portions of cannula tubing;
tightening the clamp block grasping the two portions of cannula tubing between the first non-round cannula tubing recesses and the two second non-round cannula tubing recesses; and
starting a flow of a physician recommended/controlled medication from a main source into the nasal cannula;
wherein the first and second non-round cannula tubing recesses have a width slightly greater than a diameter of the second cannula tubes, and a depth less than a radius of the second cannula tubes.

18. The method of claim 17, further including:
an independent flow sensor assembly serially connected between the main source and the standard cannula Y, the independent flow sensor assembly detecting a presence of a flow in the first cannula tubing;
a first circuit in the flow sensor assembly processing flow data from the independent flow sensor;
wirelessly transmitting the flow data to a second circuit in the clamp body; and
wirelessly transmitting the flow data from the second circuit in the clamp body to a patient monitoring device.

19. The nasal cannula clamping system of claim 17, wherein the cannula tubing passages are preferably between 0.005 and 0.002 inches wider than the diameter of the cannula tubing and between 0.0015 and 0.0045 shallower than the radius of the cannula tubing.
